(19) United States
(12) Patent Application Publication
(10) Pub. No.: US 2002/0018811 A1
(43) Pub. Date: Feb. 14, 2002

(54) Application of phytosteroids(and isomers thereof), folic acid, cyanocobalamine and pyridoxine in dietetic (alimentary) fibers

(76) Inventors: Roberto Luiz Bruno Penteado, Sao Paula (BR); Marcio Falci, Sao Paula (BR)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 09/854,859

(22) Filed: May 14, 2001

(30) Foreign Application Priority Data
May 15, 2000 (BR) PI 0001794-9

(51) Int. Cl. A61K 9/28; A61K 31/714; A61K 31/575

(52) U.S. Cl. 424/474; 514/52; 514/171; 514/251

(57) ABSTRACT

It relates to an Association of chemical agents, with intended pharmacological action to prevent the risk of infarction and brain hemorrhage caused by the development of the atherosclerotic process and of the homocysteinemia resulting from ageing.

It is of the utmost importance to prevent the increase of endogenous homocysteine.

High levels of homocysteine in blood, caused by genetical error and further biologic circumstances imparts damaging consequences upon the human organism (occlusion of blood vessels, ocular modifications, osteoporosis, nervous system).

### STEROLS

Sterols are characteristic components of all natural fats and oils (animal and vegetal); it is known, however, that during the industrial process of refining, the sterols are removed, and only a small part is retained in the oil or fat destined to human consumption. Part of the sterols is removed during the phases of refining: alkaline neutralization, bleaching and deodorization. The sterols are directed to the manufacturing of soap, or, after isolation and purification, are used as raw material for producing vitamin D and hormones.

### CONSTITUTION

Sterols are secondary alcohols (group of the steroids). The sterols of the vegetal oils are known collectively as phytosterols. Sterols are acyclic substances that contain the nucleus of cyclopentanoperhydrophenantrene.

| | |
|---|---|
| Zoosterols (animal origin) | Molecular weight |

Natural Sterols

| | | |
|---|---|---|
| Cholesterol | C₂₇H₄₆O | 386.64 |
| 7 Dihydrocholesterol | C₂₇H₄₄O | 384.62 |
| Coprosterol | C₂₇H₄₈O | 388.65 |

Phytosteroids

| | | |
|---|---|---|
| Ergosterol | C₂₈H₄₄O | 396.63 |
| Stigniasterol | C₂₉H₄₈O | 412.67 |
| Betasitosterol | C₂₉H₅₀O | 414.69 |
| Campesterol | C₂₈H₄₈O | 400.66 |
| Betasitostanol | C₂₉H₅₅O | 416.71 |

Therefore, phytosteroids (and isomers thereof) refer to the objective of the present invention.

Phytosteroids
(structural formula)

| | | |
|---|---|---|
| Ergosterol | C₂₈H₄₄O | 396.63 |

Ergosterol presents three double chains.

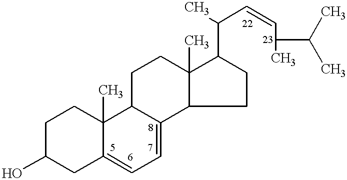

It differs from Stigmasterol for a methyl group in the lateral chain, and two double links in positions 5:6 and 7:8.

| | | |
|---|---|---|
| Stigmasterol | C₂₉H₄₈O | 412.67 |

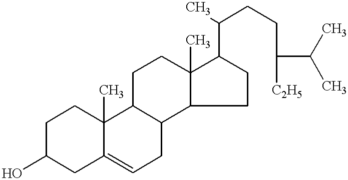

| | | |
|---|---|---|
| Betasitosterol | C₂₉H₅₀O | 414.69 |

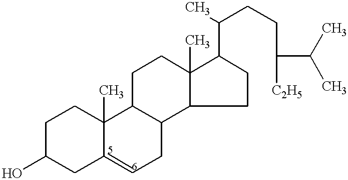

| | | |
|---|---|---|
| Campesterol | C₂₈H₄₈O | 400.66 |

Obtained from rapeseed, soya and wheat germ.

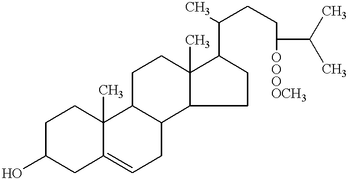

It differs from Betasitosterol for having a methyl group instead of ethyl as a substitute in the lateral chain, and from Ergosterol for not having a double link in chain 7:8.

| | | |
|---|---|---|
| Betasitostanol | C₂₉H₅₅O | 416.71 |
| (Stigmastanol) - Dihydro-β-sitostanol | | |

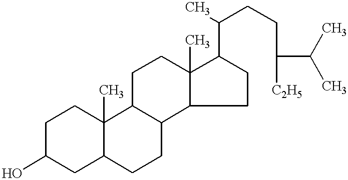

Has the same structure of betasitosterol, but having a fully saturated chain. The Phytosteroids and major isomers thereof—Ergosterol, Stigmasterol, Betasitosterol, Betasitostanol—will be used in crystallized, oily, alcoholic, aqueous, alginate (algae and derivatives thereof) and mucilaginous forms, with the purpose of conferring the desired effect, according to the prescription identified.

Biostatics, configured in the behavior of sterols in the human organism, reveals, although not sufficiently, that the complexity involved in each phase of the reaction requires the presence of different bacterial agents, salts, vitamins, enzymes, minerals and catalyzers, to satisfactorily complete the biological/physiological cycle. These basic concepts have become elementary for configuring the planning of the hypocholesteremizing action for achieving results evidencing efficiency in the reduction, under control, of cholesterol in the entero-hepatic circulation. Biodynamics may point quite precisely the participation of Sterols, particularly Betasitosterol and Betasitostanol, incorporated to dietetic fibers (alimentary), through the use of radioactive isotopes, accompanying the transformations occurred in the organisms and the secretions thereof. We sought, with this exposition of motives,—biochemical behavior—to show that a traditional stoichiometric reaction cannot prevail to reveal the evidencing chemical reactions.

The action of the phytosteroids is related to the metabolism of the lipids. Phytosteroids by oral dosage are practically not absorbed by the human intestines. They are largely similar to cholesterol, since its molecules link with the intestinal micelle that are not absorbed. The result thereof is the smaller absorption of cholesterol and the corresponding reduction of the chylomicrons. The reduction of the hepatic contents concurs and stimulates the capture of LDL and the smaller production of VLDL and apo B. Phytosteroids reduce systematically hypercholesteremia. Phytosteroids are derived from vegetal oils, thus being of natural origin and exempt of any side effects related concerning hipocholesteremia inducing drugs. The application of phytosteroids by oral dosage does not require any effort to create the habit of consumption, since they do not present any type of intolerance and do not need any adjustment of taste.

Phytosteroids, at intestinal level, will link to cholesterol (zoosterols) resulting in non absorbable micelle, and providing a high degree of deactivation of the harmful effects caused by cholesterol (zoosterols) upon the human health.

Zoosterols
(structural formula)

| | | |
|---|---|---|
| Cholesterol | C₂₇H₄₆O | 386.64 |

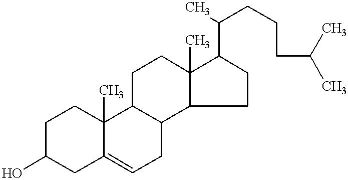

| | | |
|---|---|---|
| 7.Dehydrocolesterol | C₂₇H₄₆O | 384.62 |

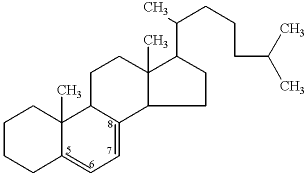

7.Dehydrocholesterol is present, in small amounts, together with cholesterol in almost every animal tissue. It differs from cholesterol by having a double link between carbons 7 and 8. Processed under ultraviolet light, , it becomes D3 vitamin (anti-rachitic).

| | | |
|---|---|---|
| Coprosterol | C₂₇H₄₈O | 388.65 |

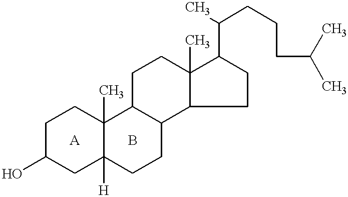

Coprosterol is found in feces, and constitutes the final product obtained by the reduction of cholesterol in the intestinal tract (through the action of intestinal bacteria). This reduction provides the deleting of the double link, the union of cycles A/B thereof is of cis configuration. It must be noted that many individuals, with cholesterol levels exceeding 240 mg/dl, do not present any defects of genetical nature, so the fact may be attributed to anomalies resulting from alimentary issues.

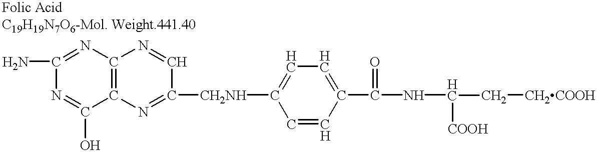

This vitamin interferes in the biosynthesis of purine and thiamine. It participates in several growth processes, and particularly in erythropoiesis. It is present in the liver, kidneys, yeasts, milk, eggs, vegetal seeds and foliage. Its name (folic) is derived from foliage. Folic acid (folacin, pteroylglutamic acid) is a composite of p-aminobenzoic acid and glutamic acid with pteridine nucleus.

Many researchers participated of its discovery, and due to the different techniques employed, resulted some factors identified by different names: vitamin M, U factor, R and S factors, norite eluate factor and streptococus latis factor (SLR).

Folic acid participates in the synthesis of the methyl (—CH₃) group in the processes of homocystheine methylation for producing methionine.

Methionine is an indispensable amino acid in the diet; it maintains the nitrogenized balance for growth and life. Its lack interrupts growth, causes multiple problems, and may lead to death.

Folic acid and vitamins B6 and B12 participate in the organic synthesis of methionine.

Another significant fact is the capacity of regenerating tetrahydrofolic acid starting from N5-methyltetrahydrofolic acid, which is the reduced form of folic acid, a catalytic self-regenerating composite, that participates in the transference of carbon.

Folic acid is known to be present in several vegetal origin foods; even so, the organic reserves are small, and mammals cannot synthesize it. Therefore the deficit of folic acid causes the reduction of the thiamine synthesis. This element participates in the formation of DNA and not of RNA; the metabolism of thiamine affects DNA, but does not compromise in any way the production of RNA.

Histidine also has its catabolism compromised; notwithstanding its clinical significance, it accumulates large quantities of the formiminoglutamic acid metabolites (FIGLU).

### B12 Vitamin

(Cyanocobalamin)

C₆₃H₈₈O₁₄N₁₄PCo—Mol. Weight.1,355.4

B12 vitamin presents a complex structure for its composites, the best known of which is cyanocobalamin. It is found in animal products and as the result of the metabolism of microorganisms.

B12 vitamin participates in the metabolism of the methyl-labile group, particularly in the biosynthesis of methionine, by the transformation of homocysteine and choline through the participation of ethanolamine.

B12 vitamin and folic acid (pteroylmonoglutamic acid) participate in a general way in the involving metabolism of synthesis and molecular interrelation of purines.

B12 vitamin is not synthesized in the human organism. The normal deposits in man are admitted as being originated by alimentary intake.

Few are the metabolic reactions unequivocally dependent onB12 vitamin. Reactions already well clarified are those of methylmalonyl-CoA mutase, that act on the isomeric conversion between methylmalonyl-CoA and succinyl-CoA, and above all the methylation of homocysteine into methionine, which produces methionine and tetrahydrofolate.

Whenever the procedure of methylation of homocysteine is not perfectly performed, a relative deficit of methionine will occur.

The conversion of methylmalonate-succinate participates in the cycles of interconversion of lipids and carbohydrates.

Studies performed in chicks and mice, administering homocysteine without the substances that provide the methyl (—CH3) group, such as methionine, betaine and choline, have shown the occurrence of disruption of the growth process of the animals.

Through the supplementation of liver extract or of B12 vitamin, the corresponding growth was resumed. Therefore it has been evidenced that B12 vitamin participates in the methylation of homocysteine.

There are other cobalamines with properties of B12 vitamin activity:

Hydroxicobalamin B12b vitamin

Anhydrous form of the latter B12a vitamin

Nitrocobalamin B12c vitamin

-B12 vitamin (sources)

The obtaining of B12 vitamin starting from bovine liver is unfeasible, due to the high final cost of the product.

To obtain 1 g of vitamin B12, 4 tons of bovine livers would be required.

Having been verified that the intestinal microorganisms synthesize B12 vitamin, the procedures of industrial production were established based on the fermentation of Streptomices griseus (the same that produces streptomycin).

Its concentration, both in the fermenting liquid and in the liver is of one part per million (1 p. p. m.).

### B6 Vitamin

### (Pyridoxine)

C₈H₁₁O₃N.HCl—(piridoxine hydrochloride) Mol. Weight.205.4

B6 vitamin presents three activity components, and is characterized by a functional group in position 4, i.e. one pyridoxine alcohol (pyridoxol), one aldehyde (pyridoxal) and one amine (pyridoxamine). These three components (composed) are collectively called pyridoxine.

The pyridoxin aldehyde (pyridoxal) and pyridoxamine also have a vitaminic activity, being designated as the vitamins of the pyridoxine group. In the tissues, it is normally esterified with phosphoric acid, and combined with enzymatic nature proteins.

Pyridoxal phosphate appears as the coenzyme of the transaminase enzymes.

Transamination in the human organism has its importance in the participation of deamination of amino acids by the transference of the amina group to α-ketoglutaric acid and the corresponding formation of glutamic acid.

The industrial and commercial product of B6 vitamin is the alcohol hydrochloride (pyridoxine hydrochloride). Thus, 1 mg of pyridoxine hydrochloride corresponds to

0.82 mg of pyridoxine (pyridoxol)

0.81 mg of pyridoxal

0.82 mg of pyridoxamine

As pyridoxal—5—phosphate, B6 vitamin acts as coenzyme of ferments that catalyze the transamination, deamination, decarboxylation, desulphydration and several divisions or syntheses of amino acids.

Deamination and desulphydration are related to the catabolism and anabolism of amino acids, particularly in the liver.

The normal metabolism of amino acids is of great importance to the disintoxication reactions, with the corresponding elimination of substances harmful to the human organism.

B6 vitamin also takes part in the maintenance of a proper level of CoA in the liver. The metabolism of fatty acids becomes reduced in the absence of B6 vitamin, resulting in problems with the metabolism of lipids.

In the metabolism of cysteine, the B6 vitamin reactions are related with the transference of sulfur from methionine to serine, resulting in cysteine. Therefore B6 vitamin is related to transamination and trans-sulfuration. The corresponding removal of sulfur from cysteine or homocysteine has the participation of desulfhydrases, with the help of pyridoxal phosphate as coenzyme.

Thus B6 vitamin plays different roles in the metabolism of amino acids:

as coenzyme for the decarboxylation and deamination of serine and treonine

in the transamination, trans-sulfuration and desulfuration of cysteine and homocysteine

in quinureninase

in the transference of amino acids to the interior of cells,

### Dietetic Fibers

(Alimentary)

The use of dietetic fibers (alimentary) in human feeding is consolidated in the more knowledgeable urban societies, just like several other products, as occurred in the decade of the twenties with refined sugar.

The dietetic fibers (alimentary) correspond to the organic residues of foodstuff (animal or vegetal) that cannot be hydrolyzed by the human digestive juices. The dietetic fibers (alimentary) with the purpose of contributing to better health are presented in the domestic market as cereal flakes, Müsli, Granola, All Bran, biscuits, seed brans in general (wheat, oats, barley, rye, plantain, etc.), mucilages, alginates, autolyses of animal products and residues from the extraction of sugar from sugar beets.

The major components are structured substances existing in the cellular walls of vegetables: cellulose, hemicellulose, pectin and lignin, as well as non-structured polysaccharides (gums, mucilages and algae polysaccharides) also present in the cellular cytoplasm.

Cellulose

Chemically, cellulose consists of linear D.glucose polymers, linked in β 1-4 glucosidics. Therefore cellulose is a linear polymer of glucose (carbohydrate), has a molecular weight ranging from 600,000 to 2,000,000. The main function of cellulose in the intestines is to link with water, one of its grams being able to retain 400 mg of water.

Hemicellulose

Hemicellulose consists of homo or heteropolysaccharide complexes, of high molecular weight. The polymer presents from 150 to 250 units of mannose. Hemicellulose may be of two types:

Hemicellulose A

containing residues of:

xylose

galactose

mannose

arabinose

glucose

Hemicellulose B (acid)

containing residues of:

uronic acids (galacturonic and glucouronic)

Like cellulose, hemicellulose is also a carbohydrate, comprising pentoses and hexoses, frequently branched. Its molecular weight varies from 10,000 to 20,000.At intestinal level hemicellulose is capable of retaining water and has the property of linking to cations.

Pectin

Pectin is found in the vegetal wall, linked to hemicellulose and intermeshed with cellulose fibers. Pectin and pectinic substances consist of a coloidal combination of polysaccharides derived from galacturonic acid polymers with chains of pentose and hexose, with a molecular weight of about 60,000 to 90,000. It produces gel by retaining water, and links to cations and organic matter, promoting the excretion of biliary acids.

Lignin

Lignin is a polymer with a molecular weight from 1,000 to 10,000, made of units of phenyl-propane linked by carbon-carbon connections. It is not a carbohydrate. In the intestinal tract it represents an inhibitor of microbial digestion of the cellular wall, since it coats cellulose and hemicellulose, and may inhibit the division of the carbohydrates of the cellular wall. Lignin is capable of combining with biliary acids, forming non absorbing complexes (unsoluble), reducing the levels of cholate in blood, and providing the transformation of hepatic cholesterol into biliary salts.

Non-structured polysaccharides (gums, mucilages and algae polysaccharides) also present in the cellular cytoplasm.

Gums

Gums are vegetal (plants) exudates having as primary units: galactose, glucuronic acid, mannose, galacturonic acid.

Mucilages

Mucilages are products of the current metabolism of vegetals, having as primary units: galactose, mannose, glucose, mannose arabinose, xylose galacturonic acid.

Gums and mucilages represent a complex of non structured polysaccharides, that may form gel in the small intestine and link with biliary acids and other organic matters. They promote the increase of volume of the fecal bolus and participate in the reduction of cholesterol, by changing the metabolism of salts.

Algae polysaccharides

Derived from primary units of mannose, xylose, glucuronic acid, they are complex polymers. The anaerobic fermentation of the polysaccharides results in energy for the development and preservation of the bacterial flora of the colon.

The microflora of the colon dehydroxylates biliary acids and hydrolizes glucoronic conjugates, and may even synthesize vitamins.

Biliary acids

The cyclic structure derives from perhydrocyclopentanofenantrene, with a lateral chain with an acid function. Hydroxylates and colanic acid are admitted as derived thereof.

The spatial structure of the cycles corresponds to androstane, coprostane and hydroxyl of lithocholic acid, to epicoprostanol.

| | | |
|---|---|---|
| Cholanic acid | C₂₄H₄₀O₂ | mol. weight 360.56 |

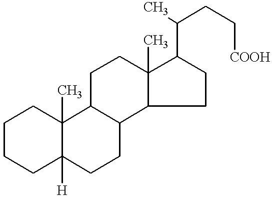

In the human bile we find:

| | |
|---|---|
| lithocholic acid containing one alcohol hydroxyl | C₂₄H₂₀O₃ |
| desoxycholic acid with two hydroxyls | C₂₄H₄₀O₄ |
| anthropocholic acid (isomer of desocycholic) | C₂₄H₄₀O₄ |
| cholic acid, with three hydroxyls | C₂₄H₄₀O₅ |

Others of lesser significance:

| | | |
|---|---|---|
| Lithocholic acid | C₂₄H₄₀O₃ | mol. weight 376.56 |

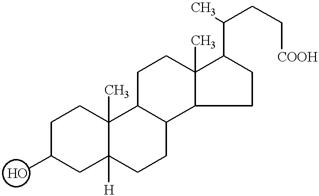

| | | |
|---|---|---|
| Desoxycholic acid | C₂₄H₄₀O₄ | mol. weight 392.56 |

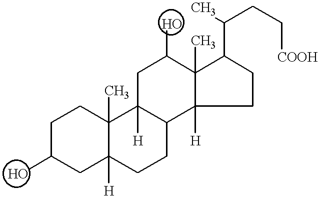

| | | |
|---|---|---|
| Cholic acid | C₂₄H₄₀O₅ | mol. weight 408.56 |

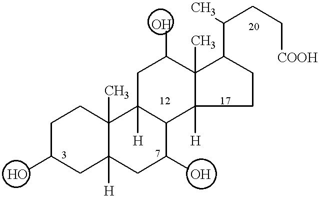

NOTE:
These acids are found in the bile, in the form of salts (mostly sodic) and others, linked to glycocoll and taurine (peptide type) attached to the carboxyl of biliary acid (amina group of aminated acid).

C₂₃H₃₉O.COOH+NH₂.CH₂—CO.OH→C₂₃H₃₉O₃, CO—NH.CH₂—COOH

Glycocholic acid is found in bile, as a sodium salt.

Cholic acid also reacts with taurine (NH₂CH₂—CH₂SO₃H), resulting taurocholic acid. Taurine is an amino acid, derived from cysteine or from the oxidation of cystine.

C₂₃H₃₉O₃CO.OH+NH₂CH₂CH₂SO₃H → C₂₃H₃₉O₃.CO.NH.CH₂.SO₃H

| | | |
|---|---|---|
| cholic acid | taurine | taurocholic acid |

| | | |
|---|---|---|
| Taurocholic acid | C₂₆H₄₅NO₇S | mol. weight 515.69 |

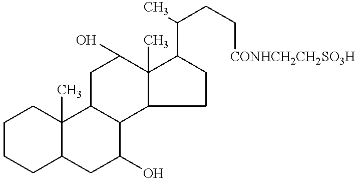

Further derivatives of cholic acid:

| | | |
|---|---|---|
| Norcholanic | C₂₃H₃₈O₂ | mol. weight 346.53 |
| obtained from ethylcholanote | | |

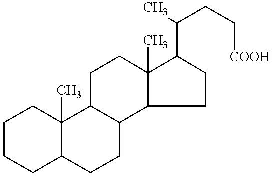

| | | |
|---|---|---|
| Ursodeoxycholanic acid | C₂₄H₄₀O₄ | mol. weight 392.56 |

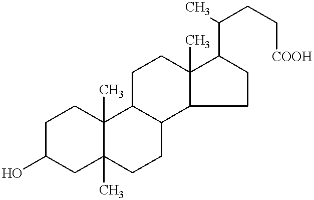

| | | |
|---|---|---|
| Chenodeoxycholanic acid | C₂₄H₄₀O₄ | mol. weight 392.56 |

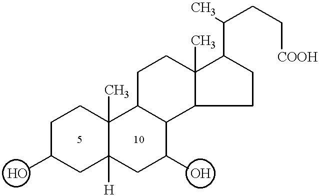

| | | |
|---|---|---|
| Dehydrocholic acid | C₂₄H₃₄O₅ | mol. weight 402.51 |

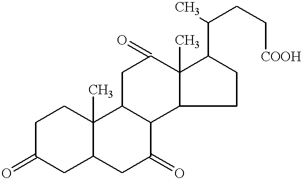

NOTE:
It is admitted that biliary acids are produced in the liver. This can be proved by the administration of tagged cholesterol (deuterium). It will be seen that part of it is oxidized, forming biliary acids. The direct synthesis of biliary salts and cholesterol must also be considered.
Biliary salts are insoluble in water and soluble in alcohol, but reduce surface tension, and as surfactants they favor the dissolution of poorly soluble or insoluble substances. Experimentally it may be seen that a solution of sodium deoxycholate increases the solubility in water of fatty acids, menthol and camphor.

Properties of great importance in the process of digestion and fat absorption.

Association of phytosteroids and polyunsaturated fatty acids (Omega 3 and Omega 6 series)

Phytosteroids (and isomers thereof and polyunsaturated fatty acids, incorporated to dietetic fibers (alimentary) will constitute a highly potentiated association of therapeutic contribution to a greater range of needs of application.

Dietetic fibers (alimentary) as excipient, with their physiological and medicamental action, dignifies the association, providing the most ample spectrum of therapeutic contribution.

The positioning of the product of the association of phytosteroids, polyunsaturated fatty acids (Omega 3 and Omega 6 series) and dietetic fibers (alimentary) will be in the direction of

Medicinal, non ethical

Alimentary complement (natural)

The presentation may be in the form of powder, sugarcoated pills, capsules, tablets, pastes, different emulsions, granulates and concentrated emulsions. The details of presentation of the product for purposes of prescription will be established according to the therapeutic application, or with the facilitated habit of ingestion. Both the form of the product and the quantities packaged will be in accordance with the specific use and the corresponding distribution for consumption.

The product technology is much too ample and well known, and may transform all the ideas and concepts prescribed by Pharmacodynamics, configured in a presentation of specific consumption, ensuring indiscussible action.

1. Application of phytosteroids (and isomers thereof), folic acid, cyanocobalamine and pyridoxine in dietetic (alimentary) fibers characterized mainly by ergosterol, stigmasterol, betasitosterol, campesterol and betasitostanol.

2. The presentation may be in the form of powder, sugarcoated pills, capsules, tablets, pastes, different emulsions, granulates and concentrated emulsions.
